(19) United States
(12) Patent Application Publication
(10) Pub. No.: US 2025/0043130 A1
(43) Pub. Date: Feb. 6, 2025

(54) METHOD FOR PRODUCING POLYHYDROXYALKANOATE, AND USE OF SAME

(71) Applicant: KANEKA CORPORATION, Osaka-shi (JP)

(72) Inventors: Naoki DEGUCHI, Takasago-shi (JP); Masaru HIRANO, Takasago-shi (JP)

(73) Assignee: KANEKA CORPORATION, Osaka-shi (JP)

(21) Appl. No.: 18/719,000

(22) PCT Filed: Dec. 14, 2022
(86) PCT No.: PCT/JP2022/045968

§ 371 Date: Jun. 12, 2024

(30) Foreign Application Priority Data
Dec. 23, 2021 (JP) 2021-209799

(51) Int. Cl. C08L 67/04; C08J 3/05

(52) U.S. Cl. CPC C08L 67/04; C08J 3/05; C08J 2367/04; C08L 2201/06; C08L 2201/54

(57) ABSTRACT

An object is to provide a PHA production method which enables efficient filtration. A PHA production method is provided, the PHA production method including: a heating step of heating an aqueous PHA suspension containing a specific additive and having a pH of 2.5 to 5.5 such that the aqueous PHA suspension has a temperature of 60° C. to 95° C.; a cooling step of cooling the aqueous PHA suspension obtained in the heating step to a specific temperature; and a filtration step of subjecting, to dead-end filtration, the aqueous PHA suspension obtained in the cooling step, with use of a filter medium having an air permeability of 0.01 cc/cm²/sec to 5.0 cc/cm²/sec, so that the above problem is solved.

### TECHNICAL FIELD

The present invention relates to a method for producing a polyhydroxyalkanoate and use of the polyhydroxyalkanoate.

### BACKGROUND ART

A polyhydroxyalkanoate (hereinafter, also referred to as a “PHA”) is known to be biodegradable.

A PHA produced by microorganisms is accumulated in the microbial cells of the microorganisms. Accordingly, in order to use a PHA as plastic, it is necessary to carry out a step of isolating a PHA from the microbial cells of the microorganisms and purifying the PHA. In the step of isolating and purifying a PHA, the microbial cells of PHA-containing microorganisms are disrupted or the biological components, excluding the PHA, are solubilized, before from an aqueous suspension thus obtained, the PHA is extracted. In this extraction, isolation operations such as, for example, centrifugation, filtration, drying are carried out.

As the PHA production method in which filtration is used, disclosed in, for example, Patent Literature 1 is a PHA production method including: a step of inoculating a fermentation medium with a species of PHA fermenter to cause fermentation; a step of subjecting a fermentation liquid to solid-liquid separation to obtain a fermentation supernatant liquid and a microbial cell precipitate; and a step of precipitating microbial cells and disrupting the cell walls of the microbial cells, and subjecting the disrupted cell walls to plate-and-frame filtration with use of a precoated filter, to obtain the PHA.

In addition, Patent Literature 2 discloses a method for recovering a PHA from the product of culturing cells and purifying the PHA, the method including a step of carrying out an acid treatment, which is a pretreatment.

### CITATION LIST

### Patent Literature

### [Patent Literature 1]

Specification of Chinese patent No. 111500650

### [Patent Literature 2]

International Publication No. WO 2015/015395

### SUMMARY OF INVENTION

### Technical Problem

However, the techniques described above are susceptible of further improvement from the viewpoint of a filtrate permeability and a leakage ratio.

An object of an aspect of the present invention is to provide a PHA production method which enables efficient filtration.

### Solution to Problem

The inventors of the present invention diligently conducted a study to solve the above problem. As a result, the inventors obtained a novel finding which indicates that by adding a specific additive and including specific steps in production of a PHA, it is possible to achieve a specific filtrate permeability and a specific leakage ratio (i.e., it is possible to carry out efficient filtration). This led to the completion of the present invention.

Thus, an aspect of the present invention is a PHA production method (hereinafter, referred to as the “present production method”) which includes a heating step of heating an aqueous PHA suspension which has a pH of 2.5 to 5.5 and which contains at least one additive selected from the group consisting of a fatty acid amide and a fatty acid, the at least one additive having a melting point of not less than 60° C., such that the aqueous PHA suspension has a temperature of 60° C. to 95° C.; a cooling step of cooling the aqueous PHA suspension obtained in the heating step such that the aqueous PHA suspension has a temperature not less than 5° C. lower than a temperature which the aqueous PHA suspension has after the heating; and a filtration step of subjecting, to dead-end filtration, the aqueous PHA suspension obtained in the cooling step, with use of a filter medium having an air permeability of 0.01 cc/cm²/sec to 5.0 cc/cm²/sec.

An aspect of the present invention is a PHA agglutinate (hereinafter, referred to as the “present PHA agglutinate”) which includes: a PHA; and at least one additive selected from the group consisting of a fatty acid amide and a fatty acid, the at least one additive having a melting point of not less than 60° C., the polyhydroxyalkanoate agglutinate containing the at least one additive in an amount of 0.3 parts by weight to 6.0 parts by weight relative to 100 parts by weight of the PHA, the PHA agglutinate having a moisture content of 25.0% to 50.0% (W.B.), the PHA agglutinate having a Feret diameter of 1 mm to 30 mm.

### Advantageous Effects of Invention

With an aspect of the present invention, it is possible to provide a PHA production method which enables efficient filtration. In particular, with the present production method, the filtration rate is high, and it is therefore possible to reduce filtration equipment costs, and possible to more efficiently carry out purification.

### DESCRIPTION OF EMBODIMENTS

The following description will discuss an embodiment of the present invention in detail. The expression “A to B” representing a numerical range means “not less than A and not more than B” unless otherwise specified herein. All of the documents cited herein are incorporated herein by reference.

### [1. Outline of Present Invention]

There was a problem of the difficulty in filtering a PHA produced within microbial cells due to the particle size of the PHA of approximately 1 μm to 2 μm. Further, since the PHA is recovered mainly by centrifugation, and the form of isolation is an aqueous suspension liquid, the PHA is recovered with a large amount of water contained. It is therefore necessary to evaporate the water contained in the aqueous suspension to separate the PHA from the water and recover the PHA. This presents a problem such as the need for a large amount of energy.

As a PHA production method in which filtration is used, the methods disclosed in, for example, Patent Literatures 1 and 2 described above are known. With the technique disclosed in Patent Literature 1, in which filtration is carried out before purification (in a state where many biological residues remain), there is the problem of an extremely low filtration rate due to the presence of impurities. Further, the inventors of the present invention carried out the replication of the technique disclosed in Patent Literature 2, and found that the pH ranged from 6 to 7. A problem found from this finding is that when filtration is carried out under this condition, a considerably large amount of resin would leak. Furthermore, the inventors found that when the mesh of a filter cloth is finer under the same condition, there is a problem of extremely low filtration rate.

To address this, the inventors of the present invention conducted a diligent study of the filtration step of PHA production from the viewpoint of improvements in a filtrate permeability and a leakage ratio. As a result, the inventors became the first to find that by adding a specific additive and including specific steps, it is possible to carry out efficient filtration. Specifically, by including: a heating step of: heating an aqueous PHA suspension containing a specific additive and having a specific pH such that the aqueous PHA suspension has a specific temperature; a cooling step of cooling the aqueous PHA suspension obtained in the heating step to a specific temperature; and a filtration step of filtering the aqueous PHA suspension obtained in the cooling step with use of a filter medium exhibiting a specific air permeability, it is possible to achieve a specific filtrate permeability and a specific leakage ratio (i.e., it is possible to carry out efficient filtration).

With the present production method, it is possible to provide a PHA production method which enables efficient filtration. In particular, with the present production method, the filtration rate is high, and it is therefore possible to reduce filtration equipment costs, and possible to more efficiently carry out purification. The present production method therefore has a great advantage in industrial production of a PHA. As used herein, the phrase “enabling efficient filtration” means that the filtrate permeability is not less than 1,400 L/m²/hr and the leakage ratio is not more than 5%.

In addition, with the configuration as described above, it is possible to reduce the quantity of heat, and time and costs, i.e., energy, required in the drying step carried out after the filtration. This enables contribution to achievement of sustainable development goals (SDGs) including, for example, Goal 7 “Ensure access to affordable, reliable, sustainable and modern energy for all”. Here is a detailed description of the present invention.

### [2. PHA Production Method]

The present production method includes the following steps:
a step (hereinafter, referred to as a step (c) or a “heat treatment step”) of heating an aqueous PHA suspension which has a pH of 2.5 to 5.5 and which contains at least one additive selected from the group consisting of a fatty acid amide and a fatty acid, the at least one additive having a melting point of not less than 60° C., such that the aqueous PHA suspension has a temperature of 60° C. to 95° C.; a step (hereinafter, referred to as a step (d′) or a “cooling step”) of cooling the aqueous PHA suspension obtained in the heating step such that the aqueous PHA suspension has a temperature not less than 5° C. lower than a temperature which the aqueous PHA suspension has after the heating; and a step (hereinafter, referred to as a step (d)) of subjecting, to dead-end filtration, the aqueous PHA suspension obtained in the cooling step, with use of a filter medium having an air permeability of 0.01 cc/cm²/sec to 5.0 cc/cm²/sec.

Further, according to an embodiment of the present invention, the present production method preferably includes, in addition to the above steps (c), (d′), and (d), at least one of the following steps.
Step (a): a step (also referred to as a “solubilizing step”) of disrupting and solubilizing cell-derived components of a microbial cell which contains a PHA, the cell-derived components excluding the PHA, the PHA having a volume median diameter of 0.5 μm to 5 μm
Step (b): a step (also referred to as a “recovery step”) of recovering an aqueous PHA suspension by centrifugation, after the step (a)
Step (c′): a step (also referred to as an “adjustment step”) of adjusting the pH of the aqueous PHA suspension obtained in the step (b) to 2.5 to 5.5
Step (e): a step (also referred to as a “drying step”) of drying the PHA obtained in step (d) at 20° C. to 80° C.
Step (f): a step (also referred to as a “redispersion step”) of redispersing, in an aqueous solvent, the PHA dried to obtain an aqueous PHA suspension which contains a PHA having a volume median diameter of 0.5 μm to 5 μm

In the present production method, the above steps are preferably carried out in the order of the steps (a), (b), (c′), (c), (d′), (d), (e), and (f). However, the order can be changed as appropriate, according to purposes. For example, the order of the steps (a) and (b) can be exchanged so that the steps are carried out in the order of the step (b) and then the step (a), and the order of the steps (c′) and (c) can be exchanged so that the steps are carried out in the order of the step (c) and then the step (c′). Further, according to purposes, each of the steps (a), (b), (c′), and (c) can be carried out twice or more. This means that an order which is, for example, the order of the step (b), the step (a), and then the step (b) or the order of the step (c′), the step (c), and then the step (c′) is possible. The steps (a), (b), (c′), (c), (d′), (d), (e), and (f) will be described below in this order for convenience. In this specification, an aqueous suspension containing at least a PHA can be expressed as an “aqueous PHA suspension” for short.

### (Step (a))

In the step (a) of the present production method, cell-derived components of a microbial cell which contains a PHA, the cell-derived components excluding the PHA, are disrupted and solubilized. By disrupting and removing, through the step (a), impurities (cell walls, protein, etc.) derived from the microbial cells, a PHA having a volume median diameter of 0.5 μm to 5 μm can be efficiently recovered from the microbial cells.

### <PHA>

As used herein, the term “PHA” is a generic term for polymers the monomer unit of which is a hydroxyalkanoic acid. The hydroxyalkanoic acid of the PHA is not particularly limited, but examples thereof include 3-hydroxybutanoic acid, 4-hydroxybutanoic acid, 3-hydroxypropionic acid, 3-hydroxypentanoic acid, 3-hydroxyhexanoic acid, 3-hydroxyheptanoic acid, and 3-hydroxyoctanoic acid. The polymer the monomer unit of which is a hydroxyalkanoic acid may be a homopolymer, or may be a copolymer which contains two or more kinds of monomer units.

More specifically, examples of the PHA include poly(3-hydroxybutyrate) (P3HB), poly(3-hydroxybutyrate-co-3-hydroxyhexanoate) (P3HB3HH), poly(3-hydroxybutyrate-co-3-hydroxyvalerate) (P3HB3HV), poly(3-hydroxybutyrate-co-4-hydroxybutyrate) (P3HB4HB), poly(3-hydroxybutyrate-co-3-hydroxyoctanoate) (P3HB3HO), poly(3-hydroxybutyrate-co-3-hydroxyoctadecanoate) (P3HB3HOD), poly(3-hydroxybutyrate-co-3-hydroxydecanoate) (P3HB3HD), and poly(3-hydroxybutyrate-co-3-hydroxyvalerate-co-3-hydroxyhexanoate) (P3HB3HV3HH). In particular, the PHA is preferably P3HB, P3HB3HH, P3HB3HV, or P3HB4HB, which is industrially easily produced.

Further, the PHA is more preferably P3HB3HH, which is a copolymer of 3-hydroxybutyric acid and 3-hydroxyhexanoic acid, from the following viewpoints: changing the composition ratio of repeating units makes it possible to change the melting point and crystallinity and thereby change the physical properties, such as Young's modulus and heat resistance, and impart a physical property intermediate between those of polypropylene and polyethylene; and P3HB3HH is easy to industrially produce as described above, and is a useful plastic in terms of physical properties.

According to an embodiment of the present invention, the composition ratio of repeating units of P3HB3HH is such that the composition ratio of a 3-hydroxybutyrate unit to a 3-hydroxyhexanoate unit is preferably 80/20 (mol/mol) to 99.9/0.1 (mol/mol) and more preferably 85/15 (mol/mol) to 97/3 (mol/mol), from the viewpoint of the balance between plasticity and strength. When the composition ratio of a 3-hydroxybutyrate unit to a 3-hydroxyhexanoate unit is not more than 99.9/0.01 (mol/mol), a sufficient plasticity is obtained, and when the composition ratio is not less than 80/20 (mol/mol), a sufficient hardness is obtained.

In the step (a), the PHA has a volume median diameter which is preferably not more than 50 times, more preferably not more than 20 times, and even more preferably not more than 10 times the volume median diameter (hereinafter referred to as a “primary particle size”) of primary particles of the PHA. When the volume median diameter of the PHA is not more than 50 times the primary particle size, the aqueous PHA suspension exhibits more excellent flowability, and the productivity of the PHA further improves accordingly.

According to an embodiment of the present invention, the volume median diameter of the PHA is, for example, preferably 0.5 μm to 5 μm, more preferably 1 μm to 4.5 μm, and even more preferably 1 μm to 4 μm, from the viewpoint of achieving excellent flowability. The volume median diameter of the PHA is measured with use of a laser diffraction/scatter particle size distribution meter LA-950 manufactured by HORIBA.

### <Microbial Cell (Microorganism)>

Microorganisms used in the step (a) are not particularly limited provided that the microorganisms can produce a PHA in the cells thereof. The microorganisms that can be used are, for example, a microorganism isolated from nature, a microorganism deposited with a depositary institution (e.g., IFO and ATCC) for microbial strains, or mutants and transformants that can be prepared from the aforementioned microorganisms. Examples of a microbial cell that produces P3HB, which is an example PHA, include Bacillus megaterium, which is the first P3HB-producing microbial cell that was discovered in 1925, and also include other natural microorganisms such as Cupriavidus necator (former classification: Alcaligenes eutrophus, Ralstonia eutropha) and Alcaligenes latus. These microorganisms are known to have a PHA accumulated within the cells thereof.

Examples of a microbial cell that produces a copolymer of hydroxybutyrate and another hydroxyalkanoate, the copolymer being an example of the PHA, include: Aeromonas caviae, which is a P3HB3HV and P3HB3HH-producing microorganism; and Alcaligenes eutrophus, which is a P3HB4HB-producing microorganism. In particular, regarding P3HB3HH, the microbial cell is more preferably Alcaligenes eutrophus AC32 strain (FERM BP-6038), which has introduced therein genes of a group of PHA synthetases, (T. Fukui, Y. Doi, J. Bacteriol., 179, pp. 4821 to 4830 (1997)), or the like, for the purpose of enhancing the productivity of P3HB3HH. Besides the above, the microbial cell may be of a genetically engineered microorganism which has introduced therein various types of PHA synthesis-related genes selected according to a desired PHA to be produced.

### <Disruption and Solubilization of Cell-Derived Components>

A method for disrupting and solubilizing cell-derived components of a microbial cell which contains a PHA, the cell-derived components excluding the PHA, in the step (a) is not particularly limited.

According to an embodiment of the present invention, the above disruption and solubilization are carried out with use of, for example, a lytic enzyme and/or a proteolytic enzyme (e.g., alkaline proteolytic enzyme).

As used herein, the term “lytic enzyme” means an enzyme that has the activity of degrading the cell wall (e.g., peptidoglycan) of a microbial cell (the activity of dissolving a microbial cell).

According to an embodiment of the present invention, the lytic enzyme is not particularly limited, but examples thereof include lysozyme, labiase, β-N-acetylglucosaminidase, an endolysin, and an autolysin. The lytic enzyme is preferably lysozyme from the viewpoint of economic advantage. One of these lytic enzymes may be used alone, or two or more of these lytic enzymes may be used in combination

As the lytic enzyme, a commercially available product can be used, and examples thereof include “Lysozyme” and “Achromopeptidase” manufactured by FUJIFILM Wako Pure Chemical Corporation.

According to an embodiment of the present invention, the optimum pH of the lytic enzyme is not particularly limited, provided that the lytic enzyme has cell wall degradation activity, but is, for example, 5.0 to 11.0, preferably 6.0 to 9.0, and more preferably 6.0 to 8.0.

According to an embodiment of the present invention, the optimum temperature of the lytic enzyme is not particularly limited, but is preferably not higher than 60° C. and more preferably not higher than 50° C., from the viewpoint of eliminating the need for undue heating to enable prevention of a thermal change (pyrolysis) of a PHA. The lower limit of the optimum temperature is not particularly limited, but is preferably not lower than room temperature (e.g., 25° C.), from the viewpoint of eliminating the need for an undue cooling operation and therefore being economical.

As used herein, the term “alkaline proteolytic enzyme” means a proteolytic enzyme having the activity of degrading protein in an alkaline environment (e.g., in a solution having a pH of 8.5).

According to an embodiment of the present invention, the alkaline proteolytic enzyme is not particularly limited, provided that the alkaline proteolytic enzyme has the activity of degrading protein in an alkaline environment, but examples thereof include serine-specific proteolytic enzymes (e.g., subtilisin, chymotrypsin, and trypsin), cysteine-specific proteolytic enzymes (e.g., papain, bromelain, and cathepsin), and aspartic acid-specific proteolytic enzymes (e.g., pepsin, cathepsin D, and HIV protease). The alkaline proteolytic enzyme is preferably a serine-specific proteolytic enzyme, and is, in particular, subtilisin (e.g., alcalase), from the viewpoint of economic advantage. One of these alkaline proteolytic enzymes may be used alone, or two or more of these alkaline proteolytic enzymes may be used in combination.

As the alkaline proteolytic enzyme, a commercially available product can be used, and examples thereof include: “Alcalase 2.5 L” manufactured by Novozyme; “Protin SD-AY10” and “Protease P ‘Amano’ 3SD” manufactured by Amano Enzyme Inc.; “Multifect PR6 L” and “Optimase PR89 L” manufactured by Danisco Japan Ltd.; “Sumizyme MP” manufactured by Shin Nihon Chemical Co., Ltd.; “Delvolase” manufactured by DSM Japan K.K.; “Biopurase OP”, “Biopurase SP-20FG”, and “Biopurase SP-4FG” manufactured by Nagase ChemteX Corporation; “Orientase 22BF” manufactured by HBI Enzymes Inc.; and “AROASE XA-10” manufactured by Yakult Pharmaceutical Industry Co., Ltd.

According to an embodiment of the present invention, the optimum pH of the alkaline proteolytic enzyme is not particularly limited, provided that the alkaline proteolytic enzyme has activity in an alkaline environment, but is, for example, 8.0 to 14.0, preferably 8.0 to 12.0, more preferably 8.0 to 10.0, even more preferably 8.0 to 9.0, and most preferably 8.5.

According to an embodiment of the present invention, the optimum temperature of the alkaline proteolytic enzyme is not particularly limited, but is preferably not higher than 60° C., and more preferably not higher than 50° C., from the viewpoint of eliminating the need for undue heating to enable prevention of a thermal change (pyrolysis) of a PHA. The lower limit of the optimum temperature is not particularly limited, but is preferably not lower than room temperature (e.g., 25° C.), from the viewpoint of eliminating the need for an undue cooling operation and therefore being economical.

According to an embodiment of the present invention, the disruption and solubilization of the cell-derived components in the step (a) can be carried out with use of lysozyme and alcalase in combination.

A time during which the above enzyme treatment is carried out in the step (a) can vary according to the type, pH, temperature, etc. of an enzyme, but is, for example, one hour to eight hours, and preferably two hours to six hours.

A solvent (the “solvent” may also be referred to as an “aqueous medium”) of the aqueous PHA suspension in the present production method may be water or a mixed solvent of water and an organic solvent. The mixed solvent contains an organic solvent compatible with water in a concentration which is not particularly limited, provided that the concentration is not higher than the aqueous solubility of the organic solvent to be used. The organic solvent compatible with water is not particularly limited, but examples thereof include: alcohols such as methanol, ethanol, 1-propanol, 2-propanol, 1-butanol, 2-butanol, iso-butanol, pentanol, hexanol, and heptanol; ketones such as acetone and methyl ethyl ketone; ethers such as tetrahydrofuran and dioxane; nitriles such as acetonitrile and propionitrile; amides such as dimethylformamide and acetamide; dimethyl sulfoxide; pyridine; and piperidine. In particular, the organic solvent compatible with water is preferably methanol, ethanol, 1-propanol, 2-propanol, 1-butanol, 2-butanol, iso-butanol, acetone, methyl ethyl ketone, tetrahydrofuran, dioxane, acetonitrile, propionitrile, or the like, in terms of being easily removed. Further, the organic solvent compatible with water is more preferably methanol, ethanol, 1-propanol, 2-propanol, butanol, acetone, or the like in terms of being easily available. Furthermore, the organic solvent compatible with water is particularly preferably methanol, ethanol, and acetone. Note that the aqueous medium of the aqueous PHA suspension is allowed to contain another solvent, a component derived from a microbial cell, a compound produced during purification, and the like, provided that the spirit of the present invention is not impaired.

The aqueous medium of the aqueous PHA suspension in the present production method preferably contains water. The water content of the aqueous medium is preferably not less than 5% by weight, more preferably not less than 10% by weight, even more preferably not less than 30% by weight, and particularly preferably not less than 50% by weight.

### (Other Steps)

According to an embodiment of the present invention, the present production method may include the following steps before the step (a).

<Step (a1)>

A step (a1) is a step of culturing a microbial cell which contains a PHA.

A microbial cell used in the step (a1) is, for example, a microbial cell described in the section <Microbial cell (microorganism)> above.

In the step (a1), a method for culturing the microbial cell is not particularly limited, but examples thereof include the method described in paragraphs [0041] to [0048] of the International Publication No. WO 2019/142717.

<Step (a2)>

A step (a2) is a step of inactivating microbial cells obtained in the step (a1). In this step, the microbial cells obtained in the step (a1) are inactivated, and an inactivated culture solution is obtained.

In the step (a2), a method for the inactivation is not particularly limited, but examples thereof include a method of heating and stirring the culture solution which contains P3HA-containing microbial cells at the temperature of the culture solution of 60° C. to 70° C. for seven hours.

<Step (a3)>

A step (a3) is a step of adjusting the concentration and pH of the inactivated culture solution obtained in the step (a2). The step (a3) is mainly carried out in a case where the viscosity of the inactivated culture solution obtained in the step (a2) is high, and the concentration and pH of the inactivated culture solution are adjusted so that the viscosity of the inactivated culture solution is reduced. Carrying out the step (a3) facilitates the solubilization in the step (a).

A method for adjusting the concentration and pH of an inactivated culture solution in the step (a3) is not particularly limited, but is carried out by any method used in the art. For example, by the addition of hydrogen peroxide, etc. to the inactivated culture solution, the concentration of the inactivated culture solution can be adjusted. Further, examples of a method for adjusting the pH include a method of adding a basic compound to the inactivated culture solution. The basic compound is not particularly limited, but is preferably an alkali metal hydroxide or an alkaline-earth metal hydroxide, and more preferably sodium hydroxide. One of the basic compounds may be used alone, or two or more of the basic compounds may be used in combination.

### (Step (b))

In the step (b) of the present production method, an aqueous PHA suspension is recovered by centrifugation after the step (a). With the step (b), it is possible to remove impurities (cell walls, protein, etc.) derived from the microbial cells contained in the aqueous PHA suspension.

In the step (b), the recovery of the aqueous PHA suspension is carried out by any centrifugation method known in the art. The method of centrifugation is not particularly limited, but examples thereof include centrifugation with use of a sedimentation centrifuge, a centrifugal dewaterer, or the like.

Examples of the sedimentation centrifuge include sedimentation centrifuges of a separator type (such as disc type, self-cleaning type, nozzle type, screw decanter type, or skimming type), a cylindrical type, and a decanter type. According to a sediment component discharging method, each of the sedimentation centrifuges is categorized into a batch-wise or continuous sedimentation centrifuge. Similarly, the centrifugal dewaterer is categorized into a batch-wise or continuous centrifugal dewaterer. By using these devices, it is possible to separate a sediment containing a PHA from a culture solution component with use of a specific gravity difference.

Since the amount of impurities which are to remain in an end product depends mainly on the steps (a) and (b), it is preferable to reduce the impurities to the smallest possible amount. As a matter of course, in some uses, the impurities are allowed to be mixed to the extent that the physical properties of the end product are not impaired. However, in a case where a highly pure PHA is required, such as the case of a medical use, it is preferable to reduce the impurities to the smallest possible amount. An index of the degree of purification in reducing the impurities can be, for example, the amount of PHA surface adhesion protein contained in the aqueous PHA suspension. The amount of such protein with respect to the weight of the PHA is not more than 2,000 ppm, preferably not more than 1,900 ppm, even more preferably not more than 1,800 ppm, and most preferably not more than 1,700 ppm. The amount of PHA surface adhesion protein in the aqueous PHA suspension falling within the above range provides the advantage that the leakage ratio is not too high. An inferred reason why this effect is brought about is that when the amount of PHA surface adhesion protein is small, PHAs easily gather.

(Step (c′))

In the step (c′), the aqueous PHA suspension recovered by centrifugation usually has a pH greater than 7. In the step (c′) of the present production method, the pH of the aqueous PHA suspension obtained in the step (b) is adjusted to 2.5 to 5.5. Carrying out the pH adjustment of the step (c′) reduces the leakage ratio in the filtration of the step (d).

In the step (c′), the pH of the aqueous PHA suspension is 2.5 to 5.5, preferably 2.5 to 5.0, more preferably 2.5 to 4.5, even more preferably 2.5 to 4.0, and particularly preferably 2.5 to 3.5. The pH of the aqueous PHA suspension falling within the above range provides the advantage of making it possible to improve the filtrate permeability without enhancing the ratio in which a PHA leaks into the filtrate in the filtration step. An inferred reason why this effect is brought about is that PHAs are not too small, and thus easily agglutinate. Further, the upper limit of the pH is preferably not more than 5.5 from the viewpoint of reducing the coloration during heating and melting of a PHA and ensuring the stability of the molecular weight during heating and/or drying, and also from the viewpoint of obtaining a PHA the coloration of which during heating and melting has been reduced and the molecular weight reduction of which during heating and/or drying has been prevented. The lower limit of the pH is preferably not more than 2.5 from the viewpoint of the resistance of a container to acid.

In the step (c′), a method for adjusting the pH is not particularly limited, but examples thereof include a method of adding acid. The acid is not particularly limited, but may be either an organic acid or an inorganic acid, and may or may not be volatile. More specifically, examples of the acid can include sulfuric acid, hydrochloric acid, phosphoric acid, and acetic acid.

According to an embodiment of the present invention, it is preferable that an additional pH adjustment should not be carried out after the pH adjustment in the step (c) and until after the step (d) is carried out.

### (Step (c))

In the step (c) of the present production method, the aqueous PHA suspension which has a pH of 2.5 to 5.5 and which contains at least one additive selected from the group consisting of a fatty acid amide and a fatty acid, the at least one additive having a melting point of not less than 60° C., is heated so as to have a temperature of 60° C. to 95° C. With the step (c), it is possible to increase the filtrate permeability during filtration.

In the step (c), the aqueous PHA suspension contains at least one additive selected from the group consisting of a fatty acid amide and a fatty acid, the at least one additive having a melting point of not less than 60° C.

In the step (c), the melting point of the additive is not less than 60° C., preferably not less than 65° C., more preferably not less than 70° C., even more preferably not less than 75° C., and particularly preferably not less than 80° C. The melting point of the additive falling within the above range provides the advantage of an improved filtrate permeability. An inferred reason why this effect is brought about is that the additive melts in portions the temperatures of which become high in a localized manner, and acts as a binder. The melting point is measured according to the method described in Examples.

The fatty acid amide having a melting point of not less than 60° C. is not particularly limited, but examples thereof include erucic acid amide (melting point: 80° C.), behenic acid amide (melting point: 110° C.), oleic acid amide (melting point: 73° C.), stearic acid amide (melting point: 106° C.), palmitic acid amide (melting point: 103° C.), N-stearylerucic acid amide (melting point: 72° C.), ethylenebisstearic acid amide (melting point: 145° C.), and ethylenebisoleic acid amide (melting point: 119° C.). Among these fatty acid amides, erucic acid amide (melting point: 80° C.) and behenic acid amide (melting point: 110° C.) are particularly preferable from the viewpoint of being light-colored. One of these fatty acid amides may be used alone, or two or more of these fatty acid amides may be used.

The fatty acid having a melting point of not less than 60° C. is not particularly limited, but examples thereof include stearic acid (melting point: 70° C.), palmitic acid (melting point: 63° C.), and behenic acid (melting point: 82° C.). Among these fatty acids, stearic acid is particularly preferable from the viewpoint of price. One of these fatty acids may be used alone, or two or more of these fatty acids may be used.

In the step (c), the additive is contained in an amount which is preferably 0.3 parts by weight to 6.0 parts by weight, more preferably 0.35 parts by weight to 5.8 parts by weight, and even more preferably 0.4 parts by weight to 5.6 parts by weight, relative to 100 parts by weight of the PHA. The additive content falling within the above range provides the advantage of an improved filtrate permeability.

In the step (c), the aqueous PHA suspension is heated in a heat treatment so as to have a temperature which is 60° C. to 95° C., preferably 65° C. to 93° C., and more preferably 70° C. to 91° C. When the temperature of the aqueous PHA suspension falls within the above range, it is possible to increase the filtrate permeability during filtration to a greater degree.

A method of the heat treatment in the step (c) is not particularly limited, but examples thereof include (i) a method of using steam to warm a container holding the aqueous PHA suspension, (ii) a method of using oil to warm a container holding the aqueous PHA suspension, and (iii) a method of directly putting steam into the aqueous PHA suspension. The temperature of the steam in the above methods (i) and (iii) and the temperature of the oil in the above method (ii) are not particularly limited provided that the temperatures cause the aqueous PHA suspension in the step (c) to have a temperature of 60° C. to 95° C., but are, for example, 95° C. to 160° C. in that when the temperatures are higher, the time required for the temperature of the aqueous PHA suspension to increase is shorter, and it is possible to increase the temperature in small-scale equipment.

(Step (d′))

In the step (d′) of the present production method, the aqueous PHA suspension obtained in the step (c) is cooled so as to have a temperature not less than 5° C. lower than a temperature which the aqueous PHA suspension has after the heating. According to an embodiment of the present invention, in the step (d′), the aqueous PHA suspension obtained in the step (c) is cooled so as to have a temperature which is preferably not less than 8° C. lower, more preferably not less than 10° C. lower, even more preferably not less than 12° C. lower, and particularly preferably not less than 15° C. lower, than the temperature which the aqueous PHA suspension has after the heating. A method for the cooling is not particularly limited, but examples thereof include cooling the aqueous PHA suspension via a cooling device and allowing the aqueous PHA suspension to cool.

Specifically, the temperature of the aqueous PHA suspension in the step (d′) is not particularly limited, but is preferably 20° C. to 90° C., more preferably not less than 20° C. and less than 90° C., even more preferably 20° C. to 85° C., particularly preferably 20° C. to 80° C., and most preferably 20° C. to 75° C. Note that such a temperature of the aqueous PHA suspension is preferably maintained until the filtration step (step (d)). That is, the temperature of the aqueous PHA suspension in the step (d′) is preferably equal to the temperature (temperature during filtration) of the aqueous PHA suspension in the step (d).

### (Step (d))

In the step (d) of the present production method, the aqueous PHA suspension obtained in the cooling step (step (d′)) is subjected to dead-end filtration with use of a filter medium having an air permeability of 0.01 cc/cm²/sec to 5.0 cc/cm²/sec. In the step (d), the amount of PHA surface adhesion protein in the aqueous PHA suspension is not more than 2,000 ppm, and the liquid density of the aqueous PHA suspension in the filtration step is 0.5 g/mL to 1.08 g/mL. With the step (d), PHAs having a constant volume median diameter and a constant moisture content are obtained.

In this specification, the amount (cc) of air passing through a unit area (cm²) of a filter medium per second is referred to as an air permeability. In the step (d), the air permeability is 0.01 cc/cm²/sec to 5.0 cc/cm²/sec, preferably 0.1 cc/cm²/sec to 4.0 cc/cm²/sec, more preferably 0.2 cc/cm²/sec to 3.5 cc/cm²/sec, even more preferably 0.3 cc/cm²/sec to 3.0 cc/cm²/sec, and particularly preferably 0.4 cc/cm²/sec to 2.5 cc/cm²/sec. The air permeability falling within the above range provides the advantage of a low ratio in which a PHA leaks into the filtrate. The air permeability in the filtration step of the present production method is measured by the method described in Examples.

In the step (d), the liquid density of the aqueous PHA suspension is 0.50 g/mL to 1.08 g/mL, preferably 0.55 g/mL to 1.05 g/mL, more preferably 0.60 g/mL to 1.02 g/mL, and even more preferably 0.65 g/mL to 1.00 g/mL. The liquid density of the aqueous PHA suspension falling within the above range provides the advantage of a high filtrate permeability and a low moisture content in the PHA agglutinate. An inferred reason why the filtrate permeability decreases when the liquid density is low is that the aqueous PHA suspension has an increased viscosity through inclusion of air, and the air and a PHA interact with each other so that the viscosity is increased. The liquid density of the aqueous PHA suspension can be adjusted by, for example, inclusion of air. When the amount of the air is increased, the liquid density of the aqueous PHA suspension decreases. When the amount of the air is reduced, the liquid density of the aqueous PHA suspension increases.

The filter medium used in the step (d) is not particularly limited, but can be selected from among various materials such as, for example, paper, filter cloth (woven cloth, non-woven cloth), screens, sintered plates, porcelain filters, polymer membranes, punching metal, and wedge wires. A filter cloth is preferably used from the viewpoint of price and ease of cleaning.

A filtration method in the step (d) only needs to be dead-end filtration, and is not particularly limited. Examples of the filtration method include suction filtration, pressure filtration, centrifugal filtration, and gravitational filtration. In particular, suction filtration, pressure filtration, and centrifugal filtration are preferably used from the viewpoint of the size of the device. Furthermore, due to the simplicity of the structure, suction filtration and pressure filtration are more preferably used.

As used herein, the filtrate permeability means the rate at which a filtrate passes through a PHA agglutinate and a filter medium. In the step (d) of the present production method, the filtrate permeability is preferably not less than 1,400 L/m²/hr, more preferably not less than 1,450 L/m²/hr, more preferably not less than 1,500 L/m²/hr, more preferably not less than 1,550 L/m²/hr, more preferably not less than 1,600 L/m²/hr, more preferably not less than 1,650 L/m²/hr, even more preferably not less than 1,700 L/m²/hr, and particularly preferably not less than 1,750 L/m²/hr. The filtrate permeability falling within the above range provides the advantage of making it possible to reduce operation time. The filtrate permeability being higher is more preferable. The upper limit of the filtrate permeability is not particularly limited, but is, for example, not more than 4,000 L/m²/hr. The filtrate permeability is measured by the method described in Examples.

As used herein, the leakage ratio means a ratio in which a PHA leaks to a filtrate after the filtration step. In the step (d) of the present production method, the leakage ratio is preferably not more than 5%, more preferably not more than 4%, more preferably not more than 3%, more preferably not more than 2%, more preferably not more than 1%, even more preferably not more than 0.5%, and particularly preferably not more than 0.3%. The leakage ratio falling within the above range has the advantage of making it possible to separate a PHA from water with a high quality of separation, and thereby makes it possible to recover a PHA with a high yield. The leakage ratio is measured by the method described in Examples.

As described above, the temperature (temperature during the filtration) of the aqueous PHA suspension in the step (d) is preferably equal to the temperature of the aqueous PHA suspension in the step (d′), and preferably 20° C. to 90° C., more preferably 20° C. to 85° C., even more preferably 20° C. to 80° C., and particularly preferably 20° C. to 75° C. The temperature of the aqueous PHA suspension falling within the above range provides the advantage of an increased filtrate permeability. The inferred reason for the increase in the filtrate permeability is that with an increase in temperature, the viscosity increases, and at the same time, the particle size increases.

In the step (d), the description of the “pH” made in (Step (c′)) above is employed. In addition, in the step (d), the description of the “amount of PHA surface adhesion protein contained in the aqueous PHA suspension” made in (Step (b)) above is also employed.

### (Step (e))

In the step (e) of the present production method, a PHA obtained in the step (d) is dried at 20° C. to 80° C. With the step (e), it is possible to evaporate water contained in the aqueous PHA suspension to adjust the moisture content.

A method for drying a PHA in the step (e) is not particularly limited, but examples thereof include: heating; vacuum drying; and drying at normal temperature. The method is preferably carried out by heating from the viewpoint of a moderate drying speed. A heating medium (e.g., hot air or a jacket) during the drying is preferably at 20° C. to 160° C., more preferably at 40° C. to 160° C., even more preferably at 40° C. to 150° C., and particularly preferably at 50° C. to 150° C.

### (Step (f))

In the step (f) of the present production method, the dried PHA is redispersed in an aqueous solvent so that an aqueous PHA suspension containing a PHA which has a volume median diameter of 0.5 μm to 5 μm is obtained. By carrying out the step (f) after the step (e), an aqueous PHA suspension is obtained, the aqueous PHA suspension containing a PHA which has a particle size substantially the same as the original particle size (primary particle size).

In the step (f), a method of the redispersion is not particularly limited, but is carried out by any method used in the art.

In the step (f), the volume median diameter of the PHA is not particularly limited, provided that the volume median diameter is substantially the same as the volume median diameter of the PHA in the step (a), but is, for example, preferably 0.5 μm to 5 μm, more preferably 1 μm to 4.5 μm, and even more preferably 1 μm to 4 μm.

### (3. Pha Agglutinate)

The present PHA agglutinate includes: a PHA; and at least one additive selected from the group consisting of a fatty acid amide and a fatty acid, the at least one additive having a melting point of not less than 60° C., the PHA agglutinate containing the at least one additive in an amount of 0.3 parts by weight to 6.0 parts by weight relative to 100 parts by weight of the PHA, the PHA agglutinate having a moisture content of 25.0% to 50.0% (W.B.) and having a Feret diameter of 1 mm to 30 mm. It should be noted that the present PHA agglutinate can be said to be a composition which includes: a PHA; and at least one additive selected from the group consisting of a fatty acid amide and a fatty acid, the at least one additive having a melting point of not less than 60° C., the present PHA agglutinate containing the at least one additive in an amount of 0.3 parts by weight to 6.0 parts by weight relative to 100 parts by weight of the PHA, the present PHA agglutinate having a moisture content of 25.0% to 50.0% (W.B.), the present PHA agglutinate having a Feret diameter of 1 mm to 30 mm. A PHA agglutinate may also be referred to as “PHA cake”, “filtration cake”, or “PHA filtration cake”.

The moisture content of the present PHA agglutinate is 25.0% to 50.0% (W.B.), preferably 25.5% to 49.0% (W.B.), more preferably 26.0% to 48.0% (W.B.), even more preferably 26.5% to 47.0% (W.B.), and particularly preferably 27.0% to 46.0% (W.B.). The moisture content of the present PHA agglutinate falling within the above range brings the PHA agglutinate into a solid state, not into a slurry state, and thus provides the advantage of making the PHA agglutinate easy to put into a dryer. The moisture content of the present PHA agglutinate is measured by the method described in Examples.

The Feret diameter of the present PHA agglutinate is 1 mm to 100 mm, preferably 1 mm to 70 mm, more preferably 1 mm to 50 mm, even more preferably 1 mm to 30 mm, and particularly preferably 1 mm to 10 mm. The Feret diameter of the present PHA agglutinate falling within the above range provides the advantage of advantageously transferring the present PHA agglutinate to the following process. In particular, the present PHA agglutinate having a Feret diameter of 1 mm to 30 mm provides the advantage of making it possible to reduce the scattering of fine powder. It should be noted that it is possible to size the PHA agglutinate obtained in the step (d) such that the Feret diameter falls within the above range, by a mechanical method such as a crusher or a screw, or by crush resulting from drop impact. The Feret diameter of the present PHA agglutinate is determined by capturing an image (FIG. 1) of 10 PHA agglutinates, analyzing the image by ImageJ (ver1.50), finding the respective Feret diameters of the 10 PHA agglutinates, and taking the simple average of the respective 10 Feret diameters.

As to the descriptions of the additive and the additive content in the present PHA agglutinate, the descriptions in (Step (c)) of [2. PHA production method] are employed.

According to an embodiment of the present invention, the present PHA agglutinate is produced by the present production method.

The present PHA agglutinate may contain various components which have been produced in the course of the present production method, or could not have been removed, provided that the present PHA agglutinate has the effect of the present invention.

### [4. Dried Product]

According to an embodiment of the present invention, a dried product (hereinafter, referred to as the “present dried product”) obtained by drying the PHA agglutinate described in the section [3. PHA agglutinate] is provided. The present dried product is obtained by, for example, the method described in (Step (e)) of [2. PHA production method], and differs from an extrusion.

The present dried product has a moisture content which is preferably 0.01% to 10.0% (W.B.), more preferably 0.1% to 5.0% (W.B.), and even more preferably 0.5% to 5.0% (W.B.). The moisture content of the present dried product falling within the above range provides the advantage of making it possible to ensure long-term stability of storage. The moisture content of the present dried product is measured by the method described in Examples.

The PHA, the present PHA agglutinate, and the present dried product that are obtained by the present production method can be used for various uses such as paper, a film, a sheet, a tube, a plate, a rod, a container (e.g. a bottle container), a bag, and a component.

The present invention is not limited to the embodiments, but can be altered by a skilled person in the art within the scope of the claims. Any embodiment derived by an appropriate combination of technical means disclosed in differing embodiments is within the technical scope of the present invention.

An embodiment of the present invention includes the following.

<1> A method for producing a polyhydroxyalkanoate including: a heating step of heating an aqueous polyhydroxyalkanoate suspension which has a pH of 2.5 to 5.5 and which contains at least one additive selected from the group consisting of a fatty acid amide and a fatty acid, the at least one additive having a melting point of not less than 60° C., such that the aqueous polyhydroxyalkanoate suspension has a temperature of 60° C. to 95° C.; a cooling step of cooling the aqueous polyhydroxyalkanoate suspension obtained in the heating step such that the aqueous polyhydroxyalkanoate suspension has a temperature not less than 5° C. lower than a temperature which the aqueous polyhydroxyalkanoate suspension has after the heating; and a filtration step of subjecting, to dead-end filtration, the aqueous polyhydroxyalkanoate suspension obtained in the cooling step, with use of a filter medium having an air permeability of 0.01 cc/cm²/sec to 5.0 cc/cm²/sec. <2> The method described in <1>, in which in the filtration step, a filtrate permeability is not less than 1,400 L/m²/hr, and a leakage ratio is not more than 5%.
<3> The method described in 1 or 2, in which the at least one additive is contained in an amount of 0.3 parts by weight to 6.0 parts by weight relative to 100 parts by weight of the polyhydroxyalkanoate.
<4> The method described in any one of <1> to <3>, in which the aqueous PHA suspension has a temperature of not less than 20° C. and less than 90° C. in the cooling step.
<5> A polyhydroxyalkanoate agglutinate including: a polyhydroxyalkanoate; and at least one additive selected from the group consisting of a fatty acid amide and a fatty acid, the at least one additive having a melting point of not less than 60° C., the polyhydroxyalkanoate agglutinate containing the at least one additive in an amount of 0.3 parts by weight to 6.0 parts by weight relative to 100 parts by weight of the polyhydroxyalkanoate, the polyhydroxyalkanoate agglutinate having a moisture content of 25.0% to 50.0% (W.B.), the polyhydroxyalkanoate agglutinate having a Feret diameter of 1 mm to 30 mm.
<6> A dried product obtained by drying the polyhydroxyalkanoate agglutinate described in <5>.

### EXAMPLES

The following description will discuss the present invention in more detail on the basis of Examples. However, the present invention is not limited to Examples. In Examples, “P3HB3HH” is used as the “PHA”. The word “PHA” described in Examples can therefore be read as “P3HB3HH”.

### (Measurement Method)

Measurements in Examples and Comparative Examples were carried out by the following method.

### (Air Permeability)

The air permeability was measured by the method described in JIS L 1096. Specifically, with use of a Frazier type permeameter (Permeameter P2 manufactured by Toyo Seiki Seisaku-sho, Ltd.), the amount of air suction was adjusted such that an inclined manometer indicates 125 Pa, and an air flow rate was measured in such a situation.

### (Amount of PHA Surface Adhesion Protein)

The amount of PHA surface adhesion protein was measured with use of BCA Protein Assay Kit (manufactured by Thermo Fisher Scientific Inc.). Specifically, the aqueous PHA suspension immediately before the step (c′) was put in a 15-mL falcon tube in an amount of 20 mg to 50 mg (in this amount of aqueous PHA suspension, approximately 10 mg of P3HB3HH particles were contained), and 2 mL of a reagent of the kit was added. After that, the falcon tube was shaken at 60° C. for 30 minutes. Thirty minutes after the end of shaking, the aqueous PHA suspension was cooled to 25° C., and the absorbance at a wavelength of 562 nm was measured.

### (Solid Concentration)

The solid concentrations of the PHA-containing culture solution after the inactivation and the aqueous PHA suspension after the pH adjustment were measured with use of a heat drying type moisture analyzer ML-50 (manufactured by A&D Company, Limited). The culture solution and the aqueous PHA suspension were heated at 130° C. until the rate at which the weights change fell below 0.05% (W.B.)/min, and the solid concentrations thereof were determined from the changes in the weights before and after the heating.

### (Liquid Density)

The aqueous PHA suspension immediately before the filtration step was heated to a filtration temperature, and 20 ml of the aqueous PHA suspension heated was then suctioned into a 20-mL plastic syringe (manufactured by Terumo Corporation) the weight of which was measured in advance. Next, the weight of the 20 mL of the aqueous suspension and the plastic syringe was measured, and a liquid density at the filtration temperature was calculated by dividing, by the volume (20 mL) of the liquid, a weight (g) obtained by subtracting the weight of the 20-mL plastic syringe from the measured weight.

### (Melting Point of Additive)

The melting point of the additive was measured via a DSC (differential scanning calorimeter Q20 manufactured by TA instruments). Specifically, the temperature of the additive was increased at a rate of 3° C./min, and a melting point peak of the additive was measured. In a case where there are two or more peaks, the inflection point of a peak of the two or more peaks, the peak having the largest peak area, was regarded as the melting point.

### (Heat Treatment Temperature)

As the heat treatment temperature, the temperature of a part of the aqueous PHA suspension was measured, the part being the farthest from a heat source, the aqueous PHA suspension having been brought into a fluidized state with use of a stirring blade or the like. For example, in a case of heating a container from without, the center of the container was measured, and in a case of directly putting steam into the center of a container, the temperature of the wall of the container was measured. The temperature was measured with a type-K thermocouple (AD5601A manufactured by A&D Company, Limited).

### (Filtration Temperature)

As the temperature during filtration, the temperature immediately before the aqueous PHA suspension was put into a filter was measured with use of a type-K thermocouple (AD5601A manufactured by A&D Company, Limited).

(pH of Aqueous PHA Suspension in Step (c′))

A PH meter (9652-10D manufactured by HORIBA) was used in the measurement. The pH was measured at a position in the aqueous PHA suspension, the position being the farthest from a position where acid was added, the aqueous PHA suspension having been brought into a fluidized state with use of a stirring blade or the like. For example, in a case of adding acid at the wall of a container, the pH was measured at the center of the container.

### (Filtrate Permeability in Suction Filtration)

A filter cloth was placed on a filter (kst-47 manufactured by Advantec) having an inner diameter of 47 mm. The filter was then mounted in a suction bell (2 L manufactured by SHIBATA) with the upper lid of the filter removed. A 50-mL graduated cylinder made of glass was placed in the suction bell so that the whole filtrate was accommodated in the graduated cylinder. By putting the aqueous PHA suspension into the filter while carrying out suction via a vacuum pump such that the pressure was reduced to −76 kPa, filtration was carried out. The dropping filtrate was captured on a video camera. With use of a time (hr) required for the discharged amount of the filtrate to reach 25 mL after 5 mL of the filtrate was discharged, and the cross-sectional area (m²) of the filter, the filtrate permeability (L/m²/hr) was calculated by dividing the amount (L) of the difference (25−5=20 mL) in the filtrate by the time (hr) and the cross-sectional area (m²). The calculation was carried out with the filtration area during the suction filtration in this specification regarded as the area of a circle having a diameter of 47 mm.

### (Leakage Ratio)

The weight of the filtrate obtained through the filtration step was measured. The absorbance of the filtrate was measured with use of a spectrophotometer (Jasco V-770 manufactured by JASCO Corporation), and the absorbance at a wavelength of 600 nm was measured. The PHA concentration in the filtrate was calculated with use of a calibration curve created from an aqueous PHA suspension having a known solid concentration. The leakage ratio (%) was calculated by finding the solid weight (g) of the filtrate from the PHA concentration of the filtrate and the weight (g) of the filtrate, and dividing the solid weight (g) of the filtrate by the solid weight (g) of the aqueous PHA suspension before the filtration step.

### (Moisture Contents of PHA Agglutinate and PHA Dried Product)

A PHA agglutinate obtained after the filtration and a PHA dried product obtained after the drying were measured with use of a heat drying type moisture analyzer ML-50 (manufactured by A&D Company, Limited). The PHA agglutinate and the PHA dried product were heated at 105° C. until the rate at which the weights changed fell below 0.05% (W.B.)/min, and the moisture contents of the PHA agglutinate and the PHA dried product were determined from the changes in the weights before and after the heating.

### (Volume Median Diameter)

The volume median diameter of an aqueous PHA suspension was measured with use of a laser diffraction/scatter particle size distribution meter LA-950 manufactured by HORIBA.

### (Feret Diameter)

The Feret diameter of the present PHA agglutinate was determined by capturing an image (FIG. 1) of 10 PHA agglutinates, analyzing the image by ImageJ (ver1.50), finding the respective Feret diameters of the 10 PHA agglutinates, and taking the simple average of the respective 10 Feret diameters.

### Example 1

### (Preparation of Microbial Cell Culture Solution)

Ralstonia eutropha described in the International Publication No. WO 2019/142717 was cultured by a method described in paragraphs [0041] to [0048] of the same document, so that a microbial cell culture solution including microbial cells containing a PHA was obtained. Note that at present, Ralstonia eutropha is classified as Cupriavidus necator.

### (Inactivation)

The microbial cell culture solution obtained as described above was subjected to heating and stirring at the temperature of the microbial cell culture solution of 60° C. to 70° C. for seven hours for sterilization. An inactivated culture solution was thus obtained.

### (Viscosity Reduction Treatment)

To the inactivated culture solution obtained as described above, 35% by weight hydrogen peroxide (manufactured by FUJIFILM Wako Pure Chemical Corporation) was added, such that the hydrogen peroxide was 1% by weight of the inactivated culture solution. Next, a 30% aqueous sodium hydroxide was added so that the pH of the inactivated culture solution was adjusted to 11.0. By keeping on adding 30% aqueous sodium hydroxide while maintaining the solution at 60° C., the pH of the solution was maintained at 11.0 for 180 minutes. An aqueous PHA suspension was thus obtained.

### (Enzyme Treatment)

To the aqueous PHA suspension obtained as described above, 95% sulfuric acid was added, so that the pH of the aqueous PHA suspension was adjusted to 7.0±0.2. The solid concentration of the aqueous PHA suspension having sulfuric acid added thereto was measured, and found to be 30% by weight. After the addition of sulfuric acid, Lysozyme (manufactured by FUJIFILM Wako Pure Chemical Corporation), which is an enzyme that degrades sugar chains (peptidoglycan) in the cell walls, was added such that the concentration thereof in the aqueous PHA suspension was 10 ppm, and the aqueous PHA suspension was kept at 50° C. for two hours. After that, Alcalase 2.5 L (manufactured by Novozyme), which is a proteolytic enzyme, was added such that the concentration thereof in the aqueous PHA suspension was 300 ppm, and 30% sodium hydroxide was then added at 50° C., so that the pH of the aqueous PHA suspension was adjusted to 8.5 and maintained for two hours.

### (Alkaline Treatment)

Sodium dodecyl sulfate (SDS, manufactured by Kao Corporation) was added to the above enzyme-treated solution so as to be 0.3% by weight of the enzyme-treated solution. After that, the pH of the enzyme-treated solution was adjusted to 11.0±0.2 with use of an aqueous sodium hydroxide solution. Next, the enzyme-treated solution was centrifuged (4,000 G, 10 minutes), and the supernatant was then removed. A twofold concentrated aqueous PHA suspension was thus obtained. The following operation was repeated 4 times: adding, to this concentrated aqueous PHA suspension, sodium hydroxide in an amount equal to that of the removed supernatant; centrifuging the aqueous PHA suspension again (4,000 G, 10 minutes); and removing a supernatant. The concentration of PHA particle surface residual protein in the aqueous PHA suspension thus obtained was 1,000 ppm. The volume median diameter of the PHA was 2.2 μm.

### (Adjustment of pH)

The obtained aqueous PHA suspension was adjusted so as to have a solid concentration of 25% by weight, and kept at 60° C. Next, the pH was adjusted to 3.0 by the addition of 10% sulfuric acid. The liquid density was 1.0 g/mL.

### (Filtration)

To the aqueous PHA suspension, 0.5 parts by weight of erucic acid amide (EA, melting point: 80° C., product name: NEUTRON-S, manufactured by Nippon Fine Chemical Co., Ltd.) was added, and the temperature of the aqueous PHA suspension was then increased until the temperature became 90° C. in an oil bath having a temperature of 125° C., and was maintained for five minutes. After that, the aqueous PHA suspension was cooled until the temperature thereof became 60° C. in a water bath having a temperature of 63° C., and then filtered. When a filter cloth (T7104C manufactured by Yabuta Kikai Co. Ltd.) having an air permeability of 0.5 cc/cm²/sec was used, the filtrate permeability stood at 1,820 L/m²/h, and the leakage ratio was 0.0%. A PHA agglutinate thus obtained had a moisture content of 44.0% (W.B.). The PHA agglutinate had a Feret diameter of 20 mm.

### Example 2

Filtration was carried out by the same method as in Example 1, except that the amount of EA added was changed to 5.0 parts by weight. The filtrate permeability stood at 2,200 L/m²/h, and the leakage ratio was 0.0%. A PHA agglutinate thus obtained had a moisture content of 47.6% (W.B.). The PHA agglutinate had a Feret diameter of 17 mm.

### Example 3

Filtration was carried out by the same method as in Example 1, except that 0.5 parts by weight of behenic acid amide (BA, melting point: 110° C., product name: BNT-22H, manufactured by Nippon Fine Chemical Co., Ltd.) was added. The filtrate permeability stood at 2,360 L/m²/h, and the leakage ratio was 1.0%. A PHA agglutinate thus obtained had a moisture content of 44.3% (W.B.). The PHA agglutinate had a Feret diameter of 12 mm.

### Comparative Example 1

Filtration was carried out by the same method as in Example 1, except that erucic acid amide was not added. The filtrate permeability stood at 1,320 L/m²/h, and the leakage ratio was 0.1%. A PHA agglutinate thus obtained had a moisture content of 44.0% (W.B.). The PHA agglutinate had a Feret diameter of 21 mm.

### Comparative Example 2

Filtration was carried out by the same method as in Example 1, except that the temperature of a liquid was not reduced to 60° C. in the filtering, and the filtration was carried out with the liquid maintained at 90° C. The filtrate permeability stood at 930 L/m²/h, and the leakage ratio was 0.1%. A PHA agglutinate thus obtained had a moisture content of 46.9% (W.B.). The PHA agglutinate had a Feret diameter of 35 mm.

TABLE 1

| | | | | | | Heat | | | | Moisture | |
|---|---|---|---|---|---|---|---|---|---|---|---|
| | | PHA | | Melting | Additive | treatment | Filtration | | | content of | |
| | | content | | point of | content | temper- | temper- | Filtrate | Leakage | PHA | Feret |
| | | [parts by | | additive | [parts by | ature | ature | permeability | ratio | agglutinate | diameter |
| | PHA | weight] | Additive | [° C.] | weight] | [° C.] | [° C.] | (L/m²/hr) | [%] | {% (W.B.}] | [mm] |
| Example 1 | P3HB3HH | 100 | EA | 80 | 0.5 | 90 | 60 | 1820 | 0.0 | 44.0 | 20 |
| Example 2 | P3HB3HH | 100 | EA | 80 | 5.0 | 90 | 60 | 2200 | 0.0 | 47.6 | 17 |
| Example 3 | P3HB3HH | 100 | BA | 110 | 0.5 | 90 | 60 | 2360 | 1.0 | 44.3 | 12 |
| Comparative | P3HB3HH | 100 | — | — | — | 90 | 60 | 1320 | 0.1 | 44.0 | 21 |
| Example 1 | | | | | | | | | | | |
| Comparative | P3HB3HH | 100 | EA | 80 | 0.5 | 90 | 90 | 930 | 0.1 | 46.9 | 35 |
| Example 2 | | | | | | | | | | | |

### [Results]

A comparison between Examples and Comparative Example 1 shows that the aqueous PHA suspension containing a fatty acid has an increased filtrate permeability. Further, a comparison between Example 1 and Comparative Example 2 shows that the filtrate permeability of the aqueous PHA suspension containing a fatty acid greatly increases by lowering the temperature of the aqueous PHA suspension before filtration.

### INDUSTRIAL APPLICABILITY

With the present production method, it is possible to produce PHAs through an easy and simple operation. This makes it possible to advantageously use the present production method in production of PHAs. Furthermore, PHAs obtained by the present production method, and a PHA agglutinate and a dried product each of which contains the PHAs can be suitably used in the fields of agriculture, fishery, forestry, horticulture, medicine, hygiene, clothing, non-clothing, packaging, motor vehicles, and building materials, and any other fields.

1: A method for producing a polyhydroxyalkanoate, the method comprising:
heating an aqueous polyhydroxyalkanoate suspension which has a pH of from 2.5 to 5.5 and which comprises at least one additive selected from the group consisting of a fatty acid amide and a fatty acid, wherein the at least one additive has a melting point of at least 60° C., such that the aqueous polyhydroxyalkanoate suspension has a temperature of from 60° C. to 95° C.;
cooling the aqueous polyhydroxyalkanoate suspension obtained in the heating such that the aqueous polyhydroxyalkanoate suspension has a temperature not less than 5° C. lower than a temperature which the aqueous polyhydroxyalkanoate suspension has after the heating; and
subjecting the aqueous polyhydroxyalkanoate suspension obtained in the cooling to dead-end filtration using a filter medium having an air permeability of from 0.01 cc/cm²/sec to 5.0 cc/cm²/sec.

2: The method according to claim 1, wherein
in the filtration, a filtrate permeability is at least 1,400 L/m²/hr, and a leakage ratio is not more than 5%.

3: The method according to claim 1, wherein
an amount of the at least one additive is from 0.3 parts by weight to 6.0 parts by weight based on 100 parts by weight of the polyhydroxyalkanoate.

4: The method according to claim 1, wherein
the aqueous polyhydroxyalkanoate suspension has a temperature of from 20° C. to less than 90° C. in the cooling.

5: A polyhydroxyalkanoate agglutinate comprising:
a polyhydroxyalkanoate; and
at least one additive selected from the group consisting of a fatty acid amide and a fatty acid, wherein the at least one additive has a melting point of at least 60° C.,
wherein the polyhydroxyalkanoate agglutinate comprises from 0.3 parts by weight to 6.0 parts by weight of the at least one additive based on 100 parts by weight of the polyhydroxyalkanoate,
the polyhydroxyalkanoate agglutinate has a moisture content of from 25.0% to 50.0% (W.B.), and
the polyhydroxyalkanoate agglutinate has a Feret diameter of from 1 mm to 30 mm.

6: A dried product obtained by drying the polyhydroxyalkanoate agglutinate according to claim 5.

7: The method according to claim 1, wherein the melting point of the at least one additive at least 70° C.

8: The method according to claim 3, wherein the amount of the at least one additive is from 0.35 parts by weight to 5.8 parts by weight based on 100 parts by weight of the polyhydroxyalkanoate.

9: The method according to claim 3, wherein the amount of the at least one additive is from 0.4 parts by weight to 5.6 parts by weight based on 100 parts by weight of the polyhydroxyalkanoate.

10: The method according to claim 1, wherein the aqueous polyhydroxyalkanoate suspension has the temperature of from 65° C. to 93° C.

11: The method according to claim 1, wherein, in the cooling, the aqueous polyhydroxyalkanoate suspension obtained in the heating has a temperature not less than 8° C. lower than a temperature which the aqueous polyhydroxyalkanoate suspension has after the heating.

12: The method according to claim 1, wherein a temperature of the aqueous polyhydroxyalkanoate suspension obtained in the cooling is from 20° C. to 90° C. and is kept at least until the filtration.

13: The method according to claim 1, wherein a liquid density of the aqueous polyhydroxyalkanoate suspension in the filtration is from 0.5 g/mL to 1.08 g/mL.

14: The method according to claim 1, wherein the filter medium has the air permeability of from 0.1 cc/cm²/sec to 4.0 cc/cm²/sec.

15: The method according to claim 1, wherein in the filtration, a filtrate permeability is at least 1,400 L/m²/hr.

16: The method according to claim 1, wherein in the filtration, a leakage ratio is not more than 5%.

17: The method according to claim 1, wherein the at least one additive is a fatty acid amide.

18: The method according to claim 1, wherein the at least one additive is selected from the group consisting of erucic acid amide, behenic acid amide, oleic acid amide, stearic acid amide, palmitic acid amide, N-stearylerucic acid amide, ethylenebisstearic acid amide, and ethylenebisoleic acid amide.

19: The polyhydroxyalkanoate agglutinate according to claim 5, wherein the at least one additive is a fatty acid amide.

20: The polyhydroxyalkanoate agglutinate according to claim 5, wherein the at least one additive is selected from the group consisting of erucic acid amide, behenic acid amide, oleic acid amide, stearic acid amide, palmitic acid amide, N-stearylerucic acid amide, ethylenebisstearic acid amide, and ethylenebisoleic acid amide.
